(19) United States
(12) Patent Application Publication
(10) Pub. No.: US 2004/0156886 A1
(43) Pub. Date: Aug. 12, 2004

(54) Sheet-like patch agent

(76) Inventor: Yasuhisa Kose, Tosu-shi (JP)

Correspondence Address: Jane Massey, Kathleen A Tyrrell, Licata & Tyrrell, 66 East Main Street, Marlton, NJ, 08053, US

(21) Appl. No.: 10/480,695

(22) PCT Filed: Jun. 12, 2002
(86) PCT No.: PCT/JP02/05838

(30) Foreign Application Priority Data
Jun. 12, 2001 (JP) 2001-176642

(51) Int. Cl. A61K 9/70; B32B 5/02

(52) U.S. Cl. 424/449; 422/123

(57) ABSTRACT

A sheet-like patch agent, comprising a support body and an adhesive agent layer, characterized in that a tension shear adhesive strength obtained when the adhesive agent surface is overlappingly adhered on the support body surface is 0.015 to 0.07 kgf/cm² (0.00147 to 0.00686 N/mm²), whereby the sheet-like patch agent safe for skin, excellent in use feeling, and excellent in working effect in addition to cooling action to affected areas, and preventing an overlapped portion from being curled up and peeled off immediately even when taped on a finger, a wrist, an ankle, a neck, an elbow, and a knee.

### TECHNICAL FIELD

The invention relates to a sheet-like patch agent. More particularly, it is a patch agent for a poultice, which is used to the body, hip, arm, face or the like and relates to the sheet-like patch agent which can be used as cosmetics, drugs or quasi-drugs which can be taped in an appropriate size on a finger, a wrist, an ankle, a neck, an elbow and a knee and is not easily curled up and peeled off even when overlapped.

### BACKGROUND ART

As a sheet-like patch agent known have been a tape preparation or a poultice used for the treatment of backache, stiff shoulders, bruise, sprain or the like, a poultice used for healing fatigue in the foot, and a sheet-like pack agent for beauty treatments for the face or the body. For example, in JP. B. H3-16989 an aqueous adhesive composition consisting of a polyacrylic acid, a polyacrylate, a cellulose derivative, a polyalcohol and a polyvalent metal compound is disclosed, in JP. A. H8-291057 a cataplasm preparation containing no pharmaceutical ingredient, wherein a moisturizing agent selected from sodium hyalulonate, sodium chondroitin sulfate, a-lactate, pyrroridone carboxylic acid, urea, an aloe extract and a perilla leaf extract is mixed is disclosed, and in JP. A. H10-279473 a sheet preparation for foot care which aims at alleviation of feet fatigue or swelling, which achieved an inproveel effect refreshing feeling or the like by the component in the water containing adhesive layer and also is excellent in use feeling is disclosed. Also, in JP. A. 2001-031563 a drug containing taping-tape, which exerts a cooling effect or an analgesic effect by being rolled once on an effected area such as a bruise or a sprain protecting and fixing the area is disclosed.

However, although there is a considerable need to tape a finger, a wrist or an ankle by a poultice, the conventional sheet-like patch agent is immediately curled up and peeled off when applied on an effected area such as a finger, a wrist, an ankle, a neck, an elbow and a knee, and has a problem that user-satisfying is yet to be developed. Also, as a solution of making, physically hard to curl by strengthening an adhesive force of a poultice adhesive agent or changing to a lipophilic tape adhesive agent have conventionally been tried, whereas on the other hand, they have problems such as an enhanced skin irritation, such as a rubor or a eruption due to a keratin exfoliation or a malaise in use, so that a sheet-like patch agent having a desired physical property has not indeed been obtained yet.

An object of the present invention is to provide a sheet-like patch agent having solved the above conventional problems, whereby the sheet-like patch agent, which is safe for the skin, excellent in use feeling, and excellent in working effect in addition to cooling action to affected areas, having no fear of an overlapped portion being immediately curled up and peeled off even when taped on a finger, a wrist, an ankle, a neck, an elbow and a knee.

### DISCLOSURE OF THE INVENTION

During extensive researches to solve the above problems the inventors, focusing on a tension shear adhesive strength obtained when the surface of a support body is overlappingly adhered to the surface of an adhesive agent, continued further research, and accomplished the invention that can solve the above problems.

Namely, the invention relates to the above sheet-like patch agent comprising a support body and an adhesive agent layer, wherein the tension shear adhesive strength obtained when the adhesive agent surface is overlappingly adhered on the support body surface is 0.015 to 0.07 kgf/cm² (0.00147 to 0.00686 N/mm²).

Also, the invention relates to the above sheet-like patch agent, wherein the holding strength obtained when the adhesive agent surface is overlappingly adhered on the support body surface is not less than 10 seconds of the falling time at 100 gf (0.98N).

Further, the invention relates to the above sheet-like patch agent, wherein the weight of the support body is 20-200 g/m².

Also, the invention relates to the above sheet-like patch agent, wherein the static friction coefficient μs is 0.5-2.5.

Further, the invention relates to the above sheet-like patch agent, wherein the support body is a sheet-like material in which one or more selected from the group consisting of polyethylene, polypropylene, polyethylene terephthalate, ethylene-vinyl acetate copolymer, vinyl chloride, polyurethane, polyester, polyamide, cotton and rayon are mixed together with pulp.

Also, the invention relates to the above sheet-like patch agent, wherein the sheet-like material is a nonwoven fabric.

Further, the invention relates to the above sheet-like patch agent, wherein the support body is the sheet-like material in which rayon and pulp are mixed together, characterized in that the mixed amount of pulp in a support body composition is 10-95% by mass.

### MODE FOR CARRYING OUT THE INVENTION

Illustrative of the support body used in the invention are a film or a flexible nonwoven fabric, wherein one or more selected from the group consisting of polyethylene, polypropylene, polyethylene terephthalate, ethylene-vinyl acetate copolymer, vinyl chloride, polyurethane, polyester, polyamide, cotton and rayon are mixed together with pulp. A combination of rayon and pulp is particularly suitable, and the mixed amount of pulp in the support body composition is 10-95% by mass, preferably 20-80% by mass, and more preferably 30-70% by mass. In parallel to the amount of pulp in the support body composition being less than 30% by mass, recognized is a tendency of curling in an overlapped portion due to reduction of the affinity between the support body and the paste, in particular the tendency being remarkable in the case of less than 10% by mass. Also recognized is a tendency of a shape destruction such as a tear due to reduction of a fracture strength of the support body in parallel to the amount being more than 70% by mass, is observed, in particular the tendency being remarkable in the case of more than 95% by mass.

The fracture strength of the support body is preferably 0.5-15 kgf/50 mm (4.9-147 N/50 mm), more preferably 1-10 kgf/50 mm (9.8-98 N/50 mm). In parallel to the fracture strength of the support body being less than 1 kgf/50 mm (9.8 N/50 mm), a tendency of a shape destruction such as a tear is observed, in particular the tendency being remarkable in the case of less than 0.5 kgf/50 mm (4.9 N/50 mm). Also recognized is a tendency of inferior use feeling (pressure feeling etc.) due to a poor flexibility in parallel to the fracture strength of the support body being more than 10 kgf/50 mm (98 N/50 mm), in particular the tendency being remarkable when exceeding 15 kgf/50 mm (147 N/50 mm). Meanwhile, the measurement of the fracture strength is carried out using Tensilon RTA-100 manufactured by ORIENTEC under the crosshead moving velocity 200 mm/min., the sample width 50 mm, and the grasping space (the sample initial length) 200 mm. In addition, the test is carried out in a test room of the atmosphere 25° C.-60% Rh.

The fracture extensibility of the support body is preferably 5-400% compared with the initial length, and more preferably 10-300%. In parallel to the fracture extensibility of the support body being less than 10%, observed a tendency of shape destrucion such as a tear and inferior use feeling (pressure feeling etc.), in particular the tendency being remarkable in the case of less 5%. Also, in parallel to the fracture extensibility of the support body being more than 300%, observed is a tendency of shape destruction such as slackening or wrinkle and an inferior taping effect, in particular the tendency being remarkable in the case of exceeding 400%. Meanwhile, the measurement of the fracture strength is carried out using Tensilon RTA-100 manufactured by ORIENTEC under the crosshead moving velocity 200 mm/min., the sample width 50 mm, and the grasping space (the sample initial length) 200 mm. In addition, the test is carried out in a test room of the atmosphere 25° C.-60% Rh.

Further, the weight of the support body is preferably 20-200 g/m², and more preferably 40-120 g/m². In parallel to the weight of the support body being less than 40 g m², observed is a tendency of shape destruction such as a tear, poor appearance due to paste oozing when applied, and poor shaping, in particular the tendency being remarkable in the case of less 20 g/m². Also, in parallel to the weight of the support body being more than 120 g/m², observed is a tendency of inferior use feeling due to poor extensibility and poor flexibility of the support body, as well as an increased cost, in particular the tendency being remarkable in the case of exceeding 200 g/m².

Furthermore, the static friction coefficient μs of the support body is preferably 0.5-2.5, and more preferably 0.7-2. In parallel to the static friction coefficient μs of the support body being less than 0.7, observed is a tendency of a reduced taping effect due to the curl of the overlapped portion, in particular the tendency being remarkable in the case of less 0.5. Also, in parallel to the static friction coefficient μs of the support body being more than 2, observed is a tendency of occurrence of a line trouble due to poor collection in manufacturing and an uncomfortable feeling in use, in particular the tendency being remarkable in the case of exceeding 2.5.

Further, stability of the preparation can be secured by applying the paste on a base fabric consisting of a flexuous support body and coating the surface of this paste layer with a removable film or paper. Also, a separation line, perforation or the like for easy sticking maybe set in the removable film or paper so that the form could render easy separation as well as easy sticking.

Although the color of the support body is not particularly limited, it greatly affects the image of the preparation and improves use feeling and an activation feeling to the skin, white, skin color, yellow, red, orange, green, blue, pink, light blue, brown and the like being cited and, if desired, a shade is preferably adjusted.

Meanwhile, the sheet-like patch agent of the invention is constituted by the above support body and the base below(adhesive agent) comprising a removable film or a removable paper. That is, it consists of a constitution comprising a moisturizing agent, water, an aqueous polymer, a cross-linking agent and an antiseptic. Also, if required, pharmaceutical ingredients, skin-care ingredients, moisturizing ingredients, antioxidants, tackifiers, dissolution agents, pigments, perfumes, surfactants, UV absorbers, inorganic fillers, pH adjusting agents and the like can be mixed.

As the moisturizing agent, glycols and/or polyalcohols can be used alone or in combination. The mixing ratio of the moisturizing agent based on the total amount of the base is 1-50% by mass, preferably 5-30% by mass, and more preferably 5-25% by mass. In parallel to the mixing ratio being less than 5% by mass, a tendency is recognized of adhesiveness and agglutinativeness of the preparation, reduced water holding property and shape retaining property before use, non-uniformity of gel, reduced workability and reduced use feeling in use, in particular the tendency being remarkable in the case of less than 1% by mass. Also, in parallel to the mixing amount being more than 25% by mass, adhesiveness and agglutinativeness of the preparation, and a water holding property and a shape retaining property before use are reduced. Also, a tendency of lowering of workability and lowering of use feeling in use is observed, whereby it is unfavorable in the case of exceeding 50% by mass because the tendency is particularly remarkable. Meanwhile, the glycols in the moisturizing agent can not only be used as a dispersion-dissolution agent or a plasticizer for water-soluble polymers, moisturizing ingredients, cross-linking agents, skin-care ingredients, antiseptics and the like, they can accelerate water release or evaporation properties.

Since the glycols cited here have a polyether structure and are lower hydrophlicity due to less hydroxyl groups compared with a low molecular polyalcohol generally used, it is possible to lower a critical relative humidity of the base ingredients except water, so that using said properties more water release to the outside in use is achieved. The result is moisture given to the skin and also suppress ion of face glow and inflammation by evaporation of water to the outside depriving the heat of vaporization thereby, as well as a pleasant refreshing feeling. Also, the temperature dependency of their viscosity is small, whereby they show a stable shape retaining property regardless of any environmental change when mixed in the preparation. As the glycols having a polyether structure, polyethylene glycol of the average molecular weight 200-600, polypropylene glycol of the average molecular weight 500-3000 are preferable, and one or more of these can be mixed for use.

Also, the polyalcohols in the moisturizing agent can not only be used as a dispersion-dissolution agent or a plasticizer for water-soluble polymers, moisturizing ingredients, cross-linking agents, skin-care ingredients, antiseptics and the like, they can accelerate water release or evaporation properties. Since the polyalcohols cited here are low-molecular polyalcohols having two or three hydroxyl groups in one molecule and are excellent in hydrophlicity, it is possible to increase a critical relative humidity of the base ingredients except water, so that using said properties and inhibition of water release and evaporation to the outside in use is achieved. Propylene glycol, 1,3-butylene glycol and glycerin are preferable as the polyalcohols, and one or more of these can be mixed for use. The mixing balance between the glycols and/or the polyalcohols in these moisturizing agents and water gives an appropriate moisturizing and an adhesiveness for the skin, whereby a comfortable refreshing feeling at the time of sticking is obtained, and the use feeling after removal is remarkably improved.

As water, purified water, sterilized water or natural water is used. Water acts as a dispersion-dissolution agent for the water soluble polymers, the moisturizing ingredients, the cross-linking agents, the antiseptics and the like, and is particularly important to disperse and dissolve the glycols and polyglycols of the moisturizing agent uniformly in the preparation. Further, water itself greatly enhances use feeling during use and after use, and moves to the skin together with the moisturizing ingredients, exerting an effect of giving moisture and tension. Thus, the mixing amount of water to be added should necessarily be a large amount of 30-95% by mass, preferably 65-90% by mass, and more preferably 70-85% by mass. The content of a large amount of water in the preparation increases the relative humidity of the preparation itself, it being possible effectively to release a large amount of water to the outside, and consequently giving moisture to the skin and being able to provide a comfortable refreshing feeling by depriving the heat of vaporization by the evaporation of water to the outside. In parallel to the mixing amount of water being less than 70% by mass, there is a tendency of adhesiveness of the preparation, lower water holding capacity before use, lower workability and lower use feeling during use, in particular the tendency being remarkable in the case of less than 30% by mass. Also, when the mixing amount exceeds 85% by mass, the adhesiveness and the agglutinativeness tends to be inhibited, as well as there is a tendency of lower shape retaining property before use, in particular the tendency being remarkable when exceeding 95% by mass.

Illustrative of the water soluble polymers are gelatin, polyacrylic acids, salts thereof, partial neutralization products thereof or the like, and each can be used individually or by mixing two or more. As the salts of polyacrylate, metal salts such as sodium, lithium and potassium are preferable, and those having average degree of polymerization of 1000-100000 are expediently used. The mixing amount of these aqueous polymers used is 3-25% by mass, preferably 5-20% by mass, and more preferably 5-10% by mass. In parallel to the mixing amount becomes less than 5% by mass, it tends to lead to lower adhesiveness and agglutinativeness of the preparation, a shape retaining property, water absorption etc., as well as non-uniformity of adhesive mass, lower workability, and lower use feeling, the tendency being remarkable in the case of less than 3% by mass. Also, in parallel to the mixing amount being more than 10% by mass, it tends to lead to lack of adhesiveness and agglutinativeness of the preparation and a shape retaining property, and also excessive viscosity increase during manufacturing, non-uniformity of adhesive mass, lower workability and lower use feeling, the tendency being remarkable when exceeding 25% by mass.

As the cross-linking agents, poorly water-soluble aluminum compounds or polyfunctional epoxy compounds can be used alone or used by mixing two or more. Illustrative of the poorly water-soluble aluminum compounds are aluminum hydroxide, aluminum hydroxide gel, aluminum silicate hydrate, synthetic aluminum silicate, kaolin, aluminum acetate, aluminum lactate, aluminum stearate, magnesium metasilicate aluminate, magnesium silicate aluminate and the like, and one or more of these can be mixed for use. Use of the poorly water-soluble aluminum compounds gives gel an appropriate strength in the initial physical property with a filler effect, in addition to an inhibitory effect against skin irritancy by the antacid action and a skin astringent action by trace aluminum ions, along with this, aluminum ions dissolving into the preparation in a time course, whereby it being possible to show a function to compensate for the lowered gel caused by time dependent decomposition of the polymer and time dependent cleavage of the cross-linking part of the covalent bondings between polymer molecules. Further, the aluminum dissolution rate can be controlled by adjusting pH.

Illustrative of the polyfunctional epoxy compounds are polyethyleneglycol diglycidyl ether, ethyleneglycol diglycidyl ether, glycerin diglycidyl ether, glycerin triglycidyl ether, propyleneglycol diglycidyl ether, polyglycerol polyglycidyl ether, sorbitol polyglycidyl ether, sorbitan polyglycidyl ether, trimethylolpropane polyglycidyl ether, pentaerythrityl polyglycidyl ether, resorcinol diglycidyl ether, neopentylglycol diglycidyl ether and the like. One or more of these polyfunctional epoxy compounds can be mixed for use. Excellent water absorption and shape retaining properties can be obtained by use of the polyfunctional epoxy compounds, and they can produce covalent bondings with water soluble polymers having a carboxyl, amino or hydroxyl groups, or the like, enhancing a gel strength.

The mixing amount of these crosslinking agents used is 0.05-20% by mass, preferably 0.5-15% by mass, and more preferably 1-10% by mass. In proportion to the mixing amount being less than 1% by mass, recognized is a tendency to induce lowering of an agglutinativeness, a shape retaining property and water absorption power of the preparation, lowering of time dependent stability in the physical property of the preparation, lowering of workability, lowering of safety for the skin and lowering of use feeling, in particular the tendency being remarkable in less than 0.05% by mass. Also, In proportion to the mixing amount becomes more than 10% by mass, recognized is a tendency to induce adhesiveness, agglutinativeness, a shape retaining property, excessive increase of viscosity during manufacturing, non-uniformity of adhesive mass by gelation, lowering of workability, lowering of safety for the skin and lowering of use feeling, and in particular the tendency being remarkable when exceeding 20% by mass.

Illustrative of the antiseptics are p-hydroxybenzoic acid esters such as methylparaben, ethylparaben and propylparaben, 1,2-pentanediol, benzoic acid, benzoate, salicylate, sorbic acid, sorbate, dehydroacetate, 4-isopropyl-3-methylphenol, 2-isopropyl-5-methylphenol, phenol, hinokitiol, cresol, 2,4,4′-trichloro-2-hydroxydiphenyl ether, 3,4,4′-trichloro-carbanide, chlorobuthanol, benzalkonium chloride and benzethonium chloride, and one or more of these can be mixed for use. The p-hydroxybenzoic acid esters are preferable among these. The mixing amount used is in 0.005-10% by mass, preferably 0.01-5% by mass, and more preferably 0.01-1% by mass. In proportion to the mixing amount becomes less than 0.01% by mass, there is a tendency to induce the putrefaction of the preparation by appearance of molds or bacteria during storage and lowering of use feeling when using and after using, the tendency being remarkable in less than 0.005% by mass. Also, in proportion to the mixing amount being more than 1% by mass, there is a tendency to bring a delicate change of the adhesiveness and agglutinativeness in the preparation and influences such as use feeling and an unpleasant feeling by irritancy and antiseptic smell, the tendency being remarkable when exceeding 10% by mass.

Further, according to a use of the patch agent, the sheet-like patch agent of the invention can appropriately be mixed in a suitable amount with pharmaceutical ingredients, skin-care ingredients, moisturizing ingredients, antioxidants, adhesiveness donating agents, dissolution agents, pigments, perfumes, surfactants, UV absorbers, inorganic fillers, pH adjusting agents and the like, which are known in themselves, in addition to the above base components.

As the pharmaceutical ingredients, they are not limited particularly if they are percutaneously absorbable drugs, illustrative of the drugs being steroidal anti-inflammatory agents such as prednisolone, dexamethasone, hydrocortisone, fluocinolone acetonide, betamethasone valerate, betamethasone dipropionate, clobetasone butylate and prednisolone succinate, non-steroidal anti-inflammatory agents and ester derivatives thereof such as methyl salicylate, glycol salicylate, indomethacin, ketoprofen, diclofenac, ibuprofen, flurbiprofen, felbinac, ketlolac, loxoprofen, suprofen, pranoprofen, thiaprofen, flufenamic acid, tenidap, aspirin, actirit, mizoribine, oxaprozin, mofezolac, etodolac, auranofin and indmethacin farnesil, antiallergic agents such as tranilast, azelastine, ketotifen, ibudilast, oxatomide, emedastine and epinastine, antihistaminic agents such as diphenhydramine, chlorpheniramine, promethazine and tripelennamine, agents for central nervous system such as chlorpromazine, nirazepam, diazepam, phenobarbital and reserpine, hormones such as insulin, testosterone, norethisterone, methyltestosterone, progesterone and estrdiol, antihypertensive agents such as clonidine,reserpine, guanethidine sulfate, efonidipine, alprenol and nifedipine, cardiotonic agents such as digitoxin and digoxin, antiarrhythmic agents such as propranolol hydrochloride, procainamide hydrochloride, ajimaline, pindolol and tulobuterol hydrochloride, coronary vasodilators such as nitroglycerin, isosorbide dinitrate, papaverine hydrochloride, nifedipine, diltiazem and nicorandil, local anesthetics such as lidocaine, procaine, procaine hydrochloride, benzocaine and tetracaine, analgesics such as morphine, aspirin, codeine, acetoanilide and aminopyrine, muscle relaxants such as tizanidine, eperisone, tolperisone, inaperisone and dantorolene, antifungal agents such as acetophenylamine, nitroflazone, pentamycine, naphthiomate, miconazole, omoconazole, clotrimazole and butenafine hydrochloride, antineoplastic agents such as 5-fluorouracil, busulfan, actinomycine, bleomycine and mitomycine, urinary incontinence agents such as terodiline hydrochloride and oxybutynin hydrochloride, antiepleptic agents such as nirazepam and meprobamate, antiparkinson agents such as chlorzoxazone, levodopa, amantadine, selegiline hydrochloride, ranolazine hydrochloride and ropinirole hydrochloride, antiemetic agents such as granisetron, azasetron, ondansetron and ramosetron, agents for pollakisuria such as oxybutin, Ca antagonists such as nifedipine, psychotropic agents such as fentanyl, morphine and imipramine, antivertigo agents such as difenidol and betahistine, cardiovascular agents such as bezothiazepin, antituissive-expectorant agents such as ketotifen, tulobuterol and tranilast, ameliorants of cerebral circulation such as vinpocetine, nicergoline, nicorandil, clentiazem maleate, fasudil hydrochloride, benidipine hydrochloride and efonidipine hydrochloride, agents for cerebrovascular dementia such as docosahexaenoic acid, vinconate hydrochloride and nebracetum fumarate, agents for Alzheimer disease such as donepazil hydrochloride, amiridin hydrochloride and memantine hydrochloride, polypeptide type hormones such as luteonizing hormone-releasing hormone and thyrotropin releasing hormone, immunomodulating agents such as polysac charides, auranofin and lobenzarit, cholagogue agents such as ursodeoxy cholic acid, diuretic agents such as hydrofulmethiazide, agents for diabetes mellitus such as tolbutamide, antipodagric agents such as colchicine, smoking cessation aid agents such as nicotine, and further, vitamins, prostaglandins, exitation-analeptic agents, hypnotic-sedative agents, autonomic agents, peripheral vasodilators, or the like.

As the skin beauty ingredients, illustrative are allantoin, glycyrrhizic acid, yeast extract, dried seawater, salt, anhydrous caffeine, l-menthol, dl-menthol, dipotassium glycyrrhizinate, papain enzyme, L-arginine, arbutin, flavonoid, collagen, yogurt extract, lecithin, ellagic acid, amino acids, kojic acid, proteins, saccharides, hormones, placenta extracts such as water-soluble placenta extract, silk or silk extract, or extraction ingredients from various crude drugs such as aloe, loofah and glycyrrhia, or plant extracts such as Angelica keiskei extract, aspalathus linearis, gambir extract, avogado extract, sweet hydrangea extract, Gynosstemma pentaphyllum extract, marshmallow extract, arnica extract, almond extract, aloe extract, benzoin extract, chestnut rose extract, Japanese knotweed extract, ginkgo extract, nettle extract, iris root extract, oolong tea extract, fennel extract, turmeric extract, rose fruit extract, Siberian ginseng extract, echinacea leaf extract, green pea extract, scutellaria root extract, phenodendron bark extract, goldthread extract, milk thisle extract, Lagestroemia extract, barley extract, fermented barley extract, okura extract, hypericam extract, white dead-nettle extract, prickly restharrow extract, watercress extract, orange extract, orange flower extract, seaweed extract, persimmon tannin, pueraria extract, valerian extract, cattail extract, annual chamomile extract, annual chamomile water, wild oat extract, chinese quince extract, carrot extract, Arthemisia capllaris extract, glycyrrhiza extraction liquid, raspberry extract, ginkgo nut extract, banaba tea extract, pagoda extract, buckwheat extract, neroli extract, magnolia extract, red-berried elder extract, hibiscus extract, cowberry extract, Japanese angelica-tree extract, guavaphenone, sophora root, feather cockscomb, mucuna extract, melosuria extract, lily bulb extract, rasberry extract, lumpuyan, green tea extract, applephenone, Japanese angelica root extract, apricot extract, tea tree extract, peach extract, macademia oil, almond oil, kiwi extract, cinchona extract, cucumber extract, apricot extract, quince seed extract, gardenia extract, Veith's bamboo extract, cumin extract, Sophora flavescens extract, walnut pod extract, grape fruit extract, clematis extract, chlorella extract, mulberry extract, mulberry leaf extract, mucuna bird woodiana tutcher extract, cinnamon extract, yellow gentian extract, crane's-bill extract, Japanese raisin tree extract, coffee extract, black tea extract, nuphar extract, burdock extract, wheat germ extract, rice bran extract, fermented rice bran extract, comfery extract, asiasarum extract, saffron extract, soapwort extract, crataegus fruit extract, Japanese pepper extract, shiitake extract, Chinese foxglove extract, lithospermum extract, perilla extract, Japanese linden extract, meadowsweet extract, peony extract, Job's-tears extract, ginger extract, sweet flag root extract, white birch extract, white birch sap, Japanese honeysuckle extract, horsetail extract, stevia extract, sage extract, sage water, common ivy extract, crataegus extract, red-berried elder extract, common juniper extract, yarrow extract, peppermint extract, mallow extract, celery extract, cnidum rhizome extract, cnidum rhizome water, sialid extract, soybean extract, jujube extract, thyme extract, tea extract, tea nut extract, clove tree extract, polyporus sclerotium extract, citrus unshiu peel extract, camellia extract, centella extract, duke extract, terminalia extract, tian cha extract, bunincasae semen extract, Japanese amgelica root extract, marigold extract, Japanese amgelica root water, vegitative wasp extract, peach kernel extract, orange peel extract, corn extract, dokudami extract, tomato extract, P. tormentilla schran k. extract, fermented soybean extract, ginseng extract, garlic extract, wild rose extract, malt extract, ophiopogon tuber extract, ophiopogon tuber extract, parsley extract, distilled mint water, rugosa rose extract, witch-hazel extract, witch-hazel extraction liquid, rose extract, parietaria extract, Plectranthus japonicus extract, cypress water, white sandalwood extract, loquat leaf extract, coltsfoot extract, tuckahoe extract, butcherbroom extract, grape extract, grape water, grape leaf extract, Siebold's beech extract, prune extract, hayflower extract, loofah extract, loofah water, safflower extract, peony extract, hop extract, pine extract, jasminum sambac extract, milk thistle extract, horse chestnut extract, Chinese soapberry extract, Swertia pseudochinesis extract, murayaco enzy extract, lemon balm extract, melilot extract, peach leaf extract, bean sprouts extract, cornflower extract, cornflower water, eucalyptus extract, eucalyptus water, strawberry saxiflage extract, Citrus junos extract, lily extract, coix extract, mugwort extract, mugwort water, lavender extract, lavender water, blue-green algae extract, green tea extract, apple extract, apple water, litchi extract, lettuce extract, lemon extract, lotus flower extract, rosemary extract, rosemary water, rose water, camomile extract, logwood extract or great burnet extract, or vitamin A, vitamin C, vitamin D, vitamin E and the other vitamins, or vitamin C derivatives such as magnesium ascorbic acid phosphate, sodium ascorbic acid phosphate or ascorbic acid-2-glucoside, or the like. Further, illustrative of drugs having a skin-whitening action are diphenhydramine hydrochloride, diphenhydramine salycilate, diphenhydramine tannate, triprolidine hydrochloride, mequitazine, chlorpheniramine maleate, d-chlorpheniramine maleate, clemastine fumarate, promethazine hydrochloride, tranilast, sodium cromoglycate, ketotifen, arylsulfatase B, bufexamac, bendazac, butyl flufenamate, ibuprofen, indomethacin, aspirin, flurbiprofen, ketoprofen, piroxicam, 2-pyridinemethyl mefenamic acid, 5,6-dehydroarachidonic acid, 5,6-methano-LTA4, esculetin, eupatilin, 4-demethyl eupatilin, caffeic acid or benoxaprofen, and one or more of these can be mixed for use.

As the moisturizing can be mixed one or more of natural moisturizing factors such as aqueous succinyl kefiran solution, aqueous acetyl kefiran solution, aqueous maleyl kefiran solution, malt root extract, rose fruit extract, orange extract, orange juice, raspberry extract, kiwi extract, cucumber extract, gardenia extract, grape fruit extract, crataegus fruit extract, Japanese pepper extract, crataegus extract, common juniper extract, zizyphi fructus extract, jujube extract, duke extract, tomato extract, grape extract, loofah extract, lime juice, apple extract, apple juice, lemon extract, lemon juice, perilla extract, mulberry bark extract, marshmallow extract, horse chestnut extract, moutan bark extract, Coix lachryma-jobi extract, olive oil, oligomer, chitin, chitosan, wheat germ extract, rice bran extract, jujube extract, hyaluronic acid, vitamin A, jojoba oil, white lupin, linoleic acd, quince seed extract, royal jelly, rose extract, annual chamomile extract, aloe, carrot extract, β-carotene, red grape extract, lemon balm extract, squalane, silk protein, collagen, sodium hyaluronate, sodium bio-hyaluronate, trehalose, litchi extract, black tea extract, chlorella extract, yeast extract, soybean extract, garlic extract, mugwort extract, aloe extract, seaweed extraction liquid, trimethylglycine, saccharides such as sorbitol, L-proline and sodium pyridocarboxylate. Further, fruit extracts (fruit juices) have action effects as a perfume.

As the antioxidants can be mixed ascorbic acid, propyl gallate, butylhydroxyanisole, dibutyl hydroxy toluene, nordihydroguaiaretic acid, tocopherol, tocopherol acetate, natural vitamin E and the like.

As the tackifiers can be mixed casein, pullulan, agar, dextran, sodium alginate, soluble starch, carboxystarch, dextrin, carboxymethyl cellulose, sodium carboxymethyl cellulose, methylcellulose, ethylcellulose, hydroxyethylcellulose, polyvinyl alcohol, polyethylene oxide, polyacrylamide, polyacrylic acid, polyvinylpyrrolidone, carboxyvinyl polymer, polyvinyl ether, polymaleic acid copolymer, methoxyethylene maleicanhydride copolymer, isobutylene maleicanhydride copolymer, polyethylene imine, polyvinyl alcohol partial hydrolysate, hydoxypropylmethyl cellulose, xanthan gum, N-vinylacetamide and the like.

As the dissolution agents can be mixed benzyl alcohol, pyrrothiodecane, peppermint oil, isopropyl myristate, crotamitone and the like.

As the pigments can be mixed the officially designated pigments such as Red No.2 (amarnath), Red No.3 (erythrosine), Red No.102 (new coccine), Red No.104-1 (phloxine B), Red No.105-1 (rose bengal), Red No.106 (acid red), Yellow No.4 (tartrazine), Yellow No.5 (sunset yellow FCF), Green No.3 (fast green FCF), Blue No.1 (brilliant blue FCF), Blue No.2 (indigo carmine) and the like. As for the pigments they are not particularly limited, though they give a great influence to the preparation image and result to the improvement of use feeling and an activation feeling to the skin.

As the surfactants can be mixed anionic surfactants such as sodium dioctyl sulfosuccinate, alkylsulfatesalts, 2-ethylhexyl alkylsulfate ester sodium salt or sodium n-dodecyl benzenesulfonate, cationic surfactants such as hexadecyl trimethylammonium chloride, octadecyl dimethyl benzyl ammonium chloride or polyoxyethylene dodecyl monomethylammonium chloride, and nonionic surfactants such as polyoxyethylene stearylether, polyoxyethylene tridecylether, polyoxyethylene nonylphenylether, polyoxyethylene octylphenylether, polyoxyethylene monostearate, sorbitan monostearate, sorbitan monopalmitate, sorbitan sesquioleate, polyoxyethylene sorbitan monolaurate, polyoxyethylene sorbitan monooleate, glycerol monostearate, polyglycerin fatty acid esters, polyoxyethylene octadecyl amine or polyoxyethylene hardened castor oil.

As the UV absorbers can be mixed p-aminobenzoic acid, p-aminobenzoic acid ester, amyl p-dimethylaminobenzoate, salicylic acid ester, methyl anthranilate, umbelliferone, esculin, benzyl cinnamate, cinoxate, guaiazulene, urocanic acid, 2-(2-hydroxy-5-methylphenyl)benzotriazole, 4-methoxybenzophenone, 2-hydroxy-4-methoxy benzophenone, dioxybenzone, octabenzone, dihydroxy dimethoxybenzophenone, sulisobenzone, benzoresorcinol, octyl p-dimetylaminobenzoate, ethylhexyl p-methoxycinnamate, or the like.

As the inorganic filler can be mixed titanium oxide, talc, zinc oxide, silicate hydrate, magnesium carbonate, calcium hydrogenphosphate, magnesium silicate, diatomaceous earth, anhydrous silicic acid, bentonite or the like.

As the pH adjusting agent can be mixed acetic acid, formic acid, lactic acid, tartaric acid, oxalic acid, benzoic acid, glycolic acid, malic acid, citric acid, hydrochloric acid, nitric acid, sulfuric acid, sodium hydroxide, potassium hydroxide, methylamine, ethylamine, propylamine, dimethylamine, diethylamine, dipropylamine, trimethylamine, triethylamine, tripropylamine, monomethanolamine, monoethanolamine, monopropanolamine, dimethanolamine, diethanolamine, diprpopanolamine, trimethanolamine, triethanolamine, tripropanolamine, citrate buffer, phosphate buffer, glycine buffer, acetate buffer or the like.

The pH of the adhesive mass appropriately mixed in a suitable amount of each of the above components should be considered not to give irritancy to the skin, and the pH of the adhesive mass is preferably in the range of 4-8, more preferably 5-7.

In a process for preparing the sheet-like patch agent of the invention, the above components are uniformly mixed and/or dissolved in a stirring apparatus and spread on the above non-dyed or dyed support body, thereon a removable paper is stuck, and it is appropriately cut in a fixed shape. That is, aiming to use in a part of the face or the body, it can be processed into a shape to be well applied to the aimed parts such as the breast, back, arms, feet, hip, shoulders, elbows, neck, fingers, wrists, ankles, neck, face, nose, eye-around or the like. Further, the sheet-like patch agent is desirably preserved in a sealed bag or container until use from the view point that contamination under preservation, decrease of effectiveness by the evaporation of the volatile substances or the like is prevented.

In the following, the sheet-like patch agents of the invention are explained in more detail by the examples and the test examples. However, the invention is not in any way limited thereby.

### EXAMPLE 1

Purified water 58.25% by mass, synthetic aluminum silicate 1% by mass, titanium oxide 1% by mass, gelatin 2.5% by mass, methylparaben 0.1% by mass, propylparaben 0.05% by mass, polyvinyl alcohol 2.5% by mass, glycerin 25% by mass, polyglycerol polyglycidyl ether 0.05% by mass, polyacrylic acid 3% by mass, partially neutralized polyacrylic acid 3% by mass, glycol salicylate 1.25% by mass, l-menthol 1% by mass, tocopherol acetate 1% by mass and dl-camphor 0.3% by mass were stirred till a homogenous dispersion and dissolution mixture. Subsequently, the mixture was spread on a support body in the proportion of 1000 g/m² and was stuck with film. Then, it was cut into 5 cm×20 cm to form the sheet-like patch agent.

The support body used in the example 1 is one in the composition of rayon/pulp (pulp content 10% by mass).

### EXAMPLE 2

Except changing the support body to one in the composition of rayon/pulp (pulp content 50% by mass), the sheet-like patch agent was prepared in the same way as Example 1.

### EXAMPLE 3

Except changing the support body to one in the composition of rayon/pulp (pulp content 95% by mass), the sheet-like patch agent was prepared in the same way as Example 1.

### EXAMPLE 4

Except changing the support body to one in the composition of polyester/pulp (pulp content 20% by mass), the sheet-like patch agent was prepared in the same way as Example 1.

### EXAMPLE 5

Except changing the support body to one in the composition of polypropylene/pulp (pulp content 30% by mass), the sheet-like patch agent was prepared in the same way as Example 1.

### EXAMPLE 6

Except changing the support body to one in the composition of polyethylene/pulp (pulp content 70% by mass), the sheet-like patch agent was prepared in the same way as Example 1.

### EXAMPLE 7

Except changing the support body to one of the composition of polyester/pulp (pulp content 80% by mass), the sheet-like patch agent was prepared in the same way as Example 1.

### EXAMPLE 8

Purified water 79.02% by mass, gelatin 2.5% by mass, methylparaben 0.2% by mass, propylparaben 0.05% by mass, propylene glycol 5% by mass, glycerin 5% by mass, ethyleneglycol diglycidyl ether 0.02% by mass, partially neutralized polyacrylic acid 5% by mass, dipotassium glycyrrhizinate 0.1% by mass, tocopherol acetate 0.1% by mass and synthetic aluminum silicate 1% by mass were stirred till a homogenous dispersion and dissolution mixture. Subsequently, the mixture was spread on a support body in the proportion of 714 g/m² and was stuck with film. Then, it was cut into 5 cm×20 cm to afford the sheet-like patch agent.

The support body used in the example 8 is one in the composition of polyester/pulp (pulp content 50% by mass).

### EXAMPLE 9

Purified water 34.8% by mass, glycerin 30% by mass, methylparaben 0.1% by mass, kaolin 1% by mass, gelatin 2% by mass, sorbitan polyglycidyl ether 0.05% by mass, polyacrylic acid 4% by mass, partially neutralized polyacrylic acid 3.5% by mass, synthetic aluminum silicate 0.5% by mass, polyvinylpyrrolidone 3% by mass, polyethylene glycol 15% by mass, propylene glycol 5% by mass, ketoprofen 0.3% by mass, l-menthol 0.3% by mass, crotamiton 0.25% by mass and oxybenzone 0.2% by mass were stirred till a homogenous dispersion and dissolution mixture. Subsequently, the mixture was spread on a support body in the proportion of 500 g/m² and was stuck with film. Then, it was cut into 5 cm×20 cm to afford the sheet-like patch agent.

The support body used in the example 9 is one in the composition of polyester/pulp (pulp content 50% by mass).

### COMPARATIVE EXAMPLE 1

Except changing the support body to one in the composition of polyester 100%, the sheet-like patch agent was prepared in the same way as Example 1.

### TEST EXAMPLE 1

### Friction Coefficient Test

As for each sheet-like patch agent in the examples 1, 2, 3, 4, 5, 6, 7 and the comparative example 1, the friction coefficient test of the support bodies used was carried out. The test was carried out according to the inclination method of Japan Industrial Standards (friction coefficient test of paper and paperboard) P8147, and as the test machine was used SLIP ANGLE TYPE FRICTION ER AN (manufactured by Toyo Seiki Seisakusho Co., Ltd.). Further, the test was performed under the conditions of inclination velocity 2.7°/sec, thread weight 1 kg, thread width 60 mm and thread length 100 mm, and the calculation was made on μs=tan θ. The results are shown in Table 1.

TABLE 1

| | Static friction |
|---|---|
| Samples | coefficient μs (tanθ) |
| Example 1 | 0.512 |
| Example 2 | 1.519 |
| Example 3 | 2.480 |
| Example 4 | 0.581 |
| Example 5 | 0.724 |
| Example 6 | 1.957 |
| Example 7 | 2.303 |
| Comparative Example 1 | 0.430 |

### TEST EXAMPLE 2

### Skin Safety Test

As for each sheet-like patch agent in the examples 1, 2, 3, 4, 5, 6, 7 and the comparative example 1, the 48-hr closed patch test was carried out. Each sample was stuck on the inner part of the upper arm for 30 persons of men and women, and the irritancy degree of the skin was evaluated after 1 hr and 24 hrs from the removal. The results are shown in Table 2.

TABLE 2

| | | Judgement | | | | Total | Positive ratio (%) |
|---|---|---|---|---|---|---|---|
| | Samples | ++ | + | ± | − | (person) | Not less than ± |
| 1 hr | Example 1 | 0 | 0 | 1 | 29 | 30 | 3.3 |
| After | Example 2 | 0 | 0 | 0 | 30 | 30 | 0.0 |
| Removal | Example 3 | 0 | 0 | 0 | 30 | 30 | 0.0 |
| | Example 4 | 0 | 0 | 0 | 30 | 30 | 0.0 |
| | Example 5 | 0 | 0 | 0 | 30 | 30 | 0.0 |
| | Example 6 | 0 | 0 | 0 | 30 | 30 | 0.0 |
| | Example 7 | 0 | 0 | 0 | 30 | 30 | 0.0 |
| | Comparative | 0 | 0 | 2 | 28 | 30 | 6.7 |
| | Example 1 | | | | | | |
| 24 hrs | Example 1 | 0 | 0 | 0 | 30 | 30 | 0.0 |
| After | Example 2 | 0 | 0 | 0 | 30 | 30 | 0.0 |
| Removal | Example 3 | 0 | 0 | 0 | 30 | 30 | 0.0 |
| | Example 4 | 0 | 0 | 0 | 30 | 30 | 0.0 |
| | Example 5 | 0 | 0 | 0 | 30 | 30 | 0.0 |
| | Example 6 | 0 | 0 | 0 | 30 | 30 | 0.0 |
| | Example 7 | 0 | 0 | 0 | 30 | 30 | 0.0 |
| | Comparative | 0 | 0 | 0 | 30 | 30 | 0.0 |
| | Example 1 | | | | | | |

### TEST EXAMPLE 3

### Sensory Test

As for each sheet-like patch agent in the examples 1, 2, 3, 4, 5, 6, 7 and the comparative example 1, the sensory test was carried out using 10 subjects respectively. As for the subjects one sheet of sample was stuck for 6 hrs, and the absolute evaluation was made on each sample. Then, the subjects were let evaluate on the items (use feeling: fitness) and (curl of overlapped portion). The results are shown in Table 3 (use feeling: fitness) and in Table 4 (curl of overlapped portion).

TABLE 3
(Use feeling: Fitness)

Each 10 persons

| | | Rather | Not | Rather | |
|---|---|---|---|---|---|
| Samples | Bad | Bad | definite | good | Good |
| Example 1 | 0 | 0 | 0 | 1 | 9 |
| Example 2 | 0 | 0 | 0 | 0 | 10 |
| Example 3 | 0 | 0 | 0 | 2 | 8 |
| Example 4 | 0 | 0 | 0 | 2 | 8 |
| Example 5 | 0 | 0 | 0 | 0 | 10 |
| Example 6 | 0 | 0 | 0 | 0 | 10 |
| Example 7 | 0 | 0 | 0 | 1 | 9 |
| Comparative | 0 | 1 | 6 | 3 | 0 |
| Example 1 | | | | | |

TABLE 4
(Curl of overlapped portion)

| | Each 10 persons | | | | |
|---|---|---|---|---|---|
| | | | | Curling | |
| | | ½ | ¼ | at overlapped | No |
| Samples | Detachment | removal | removal | portion | curling |
| Example 1 | 0 | 0 | 0 | 0 | 10 |
| Example 2 | 0 | 0 | 0 | 0 | 10 |
| Example 3 | 0 | 0 | 0 | 0 | 10 |
| Example 4 | 0 | 0 | 0 | 1 | 9 |
| Example 5 | 0 | 0 | 0 | 0 | 10 |
| Example 6 | 0 | 0 | 0 | 0 | 10 |
| Example 7 | 0 | 0 | 0 | 0 | 10 |
| Comparative | 0 | 2 | 3 | 5 | 0 |
| Example 1 | | | | | |

### TEST EXAMPLE 4

### Tension Shear Strength Test when the Support Body Surface is Overlappingly Adhered to the Adhesive Agent Surface

As for each sheet-like patch agent in the examples 1, 2, 3 and the comparative example 1, the tension shear strength test was carried out. The test was carried out in principle according to JISK6850. Further, the crosshead moving velocity was made 50 mm/min, and the test piece whose adhesive part was made by pressure-adhesion with roller [load 800 gf (7.84N), 300 mm/min., one back-and-forth)] was used. The measurement was made five times for each sample, and the average value was calculated. The results were shown in Table 5.

TABLE 5

| Samples | Shear adhesive strength |
|---|---|
| Example 1 | 0.0151 kgf/cm² (0.00148 N/mm²) |
| Example 2 | 0.0342 kgf/cm² (0.00335 N/mm²) |
| Example 3 | 0.0712 kgf/cm² (0.00698 N/mm²) |
| Comparative | 0.0108 kgf/cm² (0.00106 N/mm²) |
| Example 1 | |

### TEST EXAMPLE 5

### Holding Strength when the Support Body Surface is Overlappingly Adhered to the Adhesive Agent Surface

As for each sheet-like patch agent in the examples 1, 2, 3 and the comparative example 1, the holding strength test was carried out. The test was carried out in principle according to JISZ0237. Further, the test piece whose adhesive part was made by pressure-adhesion with a roller [load 800 gf (7.84N), 300 mm/min., one back-and-forth)] was used, and the holding strength was measured three times for each sample by way of the falling time at the load of 100 gf (0.98N), and the average value was calculated. The results were shown in Table 6.

TABLE 6

| Samples | Falling time (sec) |
|---|---|
| Example 1 | 10 |
| Example 2 | 71 |
| Example 3 | Holding not for |
| | less than 5 min. |
| Comparative | 1 |
| Example 1 | |

As described above, it was found that the sheet-like patch agents of the invention were safe for skin, excellent in use feeling, and excellent in working effect in addition to cooling action to affected areas, and prevented the overlappped portion from being curled up and peeled off immediately even when taped on a finger, a wrist, an ankle, a neck, an elbow, and a knee.

Industrial Applicability

The sheet-like patch agents of the invention have the following excellent characteristics.

a. They achieve effects of both compressing effect and taping effect together.

b. They prevent an overlapped portion from being curled up and peeled off immediately even when taped on a finger, a wrist, an ankle, a neck, an elbow, and a knee, thereby they act efficiently and effectively.

c. Since they are safe for the skin, they can be utilized for skindressing and cosmetic purposes, or treatment to an injured portion.

The sheet-like patch agents of the invention are safe for skin, excellent in use feeling, and excellent in working effect in addition to cooling action to affected areas, and prevent an overlapped portion from being curled up and peeled off immediately even when taped on a finger, a wrist, an ankle, a neck, an elbow, and a knee, and therefore, they can be applied in the fields of drugs, quasi-drugs or cosmetics used for a therapy, skindressing and cosmetic purposes, and are industrially very useful.

1. A sheet-like patch agent comprising a support body and an adhesive agent layer, wherein the tension shear adhesive strength obtained when the adhesive agent surface is overlappingly adhered on the support body surface is 0.015 to 0.07 kgf/cm² (0.00147 to 0.00686 N/mm²).

2. The sheet-like patch agent according to claim 1, wherein the holding strength obtained when the adhesive agent surface is overlappingly adhered on the support body surface is not less than 10 seconds of the falling time at 100 gf (0.98N).

3. The sheet-like patch agent according to claims 1 or 2, wherein the weight of the support body is 20-200 g/m².

4. The sheet-like patch agent according to any one of claims 1 to 3, wherein the static friction coefficient μs is 0.5-2.5.

5. The sheet-like patch agent according to any one of claims 1 to 4, wherein the support body is a sheet-like material in which one or more selected from the group consisting of polyethylene, polypropylene, polyethylene terephthalate, ethylene-vinyl acetate copolymer, vinyl chloride, polyurethane, polyester, polyamide, cotton and rayon are mixed together with pulp.

6. The sheet-like patch agent according to claim 5, wherein the sheet-like material is a nonwoven fabric.

7. The sheet-like patch agent according to claim 6, wherein the support body is the sheet-like material in which rayon and pulp are mixed together, characterized in that the mixed amount of pulp in a support body composition is 10-95% by mass.
